(19) United States
(12) Patent Application Publication
(10) Pub. No.: US 2007/0081951 A1
(43) Pub. Date: Apr. 12, 2007

(54) Prevention of dental plaque and dental calculus in animals

(76) Inventor: George K. Stookey, Noblesville, IN (US)

Correspondence Address: SCHWEGMAN, LUNDBERG, WOESSNER & KLUTH, P.A., P.O. BOX 2938, MINNEAPOLIS, MN, 55402, US

(21) Appl. No.: 11/248,013

(22) Filed: Oct. 11, 2005

(51) Int. Cl. A61K 8/49; A61K 8/36

(52) U.S. Cl. 424/49; 424/442

(57) ABSTRACT

The present invention provides methods to improve the dental health of an animal, comprising inhibiting dental plaque and calculus on the teeth of a dental plaque- and calculus-forming animal, comprising contacting said teeth with a food product comprising an amount of an antimicrobial agent effective to inhibit dental plaque and inhibit the development of gingivitis in the oral soft tissue of said animal, and with or without an acidulent amount of phosphoric acid, and an amount of a polycarboxylic acid sequestering agent effective to inhibit dental calculus.

### BACKGROUND OF THE INVENTION

Virtually all domestic companion animals (dogs and cats), as well as many other animals (primates, many exotic animals), accumulate dental plaque and calculus, and can become afflicted with gingivitis that typically progresses into periodontal disease.

Dental plaque is formed as a result of the bacterial colonization of teeth. Dental plaque is a layer of bacteria on the erupted surfaces of teeth and in the gingival crevice. The presence of plaque comprising specific bacterial species can lead to the development of dental caries and periodontal disease.

Dental calculus, or tartar, is a recurring calcified deposit on the surfaces of the teeth of many animals, including domesticated dogs and cats, humans, and primates. It is generally recognized that dental calculus develops in a sequential process that involves the accumulation of dental plaque and the subsequent calcification of the plaque by saliva, which contains very high concentrations of calcium and phosphate.

Consequences of periodontal disease can include systemic infection, alveolar bone recession, tooth decay and loss, and adverse mouth and breath odors. Once formed, calculus deposits can only be removed through dental prophylaxis or other mechanical procedures. Thus, the prevention of dental plaque and dental calculus is of importance not only for cosmetic reasons, but also because of their respective roles in the development of periodontal disease, and the resultant systemic infections, alveolar bone recession, interference in the normal mastication process, tooth loss and adverse mouth and breath odors.

Since the microorganisms comprising dental plaque are recognized as the etiological agents responsible for the development of inflammation of the oral soft tissues (e.g., gingivitis), it is well accepted that measures that remove dental plaque (e.g. tooth brushing) or prevent the formation of dental plaque (e.g. antimicrobial agents) will prevent gingivitis, and thereby prevent the progression from gingivitis to periodontal disease and improve oral health. For these reasons, antimicrobial agents are often used in dentifrices and mouth rinses for humans.

Formation of dental calculus is currently prevented in four ways. First, dental calculus formation can be prevented by meticulous, daily removal of dental plaque prior to its calcification. Second, dental calculus formation can be prevented by the daily application of crystal growth inhibitors that interfere with the calcification of dental plaque by saliva. Third, dental calculus formation may be prevented by the use of sodium hexametaphosphate. U.S. Pat. No. 5,296,217, issued on Mar. 22, 1994, and U.S. Pat. No. 5,618,518, issued on Apr. 8, 1997, disclose that sodium hexametaphosphate can prevent or reduce calculus build-up when applied as a coating to dry pet foods, pet chew products, or when mixed with moist foods. Although sodium hexametaphosphate is incorporated into some commercially-available dry dog and cat diets, and snack foods, to prevent dental calculus formation, many food products are incompatible with sodium hexametaphosphate. When sodium hexametaphosphate is applied as a final coating over a food product which has been coated with or contains phosphoric acid, a chemical conversion occurs and the ability of the hexametaphosphate to prevent calculus formation is destroyed. Thus, incorporation of sodium hexametaphosphate into animal foods previously coated with or containing phosphoric acid actually negates the beneficial effects of sodium hexametaphosphate.

Fourth, dental calculus formation may be prevented by the use of malic acid. U.S. Pat. No. 6,080,419, issued on Jun. 27, 2000, discloses that malic acid can prevent, reduce, or inhibit calculus build-up when applied as a coating to dry pet foods containing phosphoric acid.

Unfortunately, the hexametaphosphate and the malic acid technology have demonstrated no significant effect upon dental plaque or gingivitis.

Therefore, a need exists for agents to effectively control, reduce, and/or inhibit formation of dental plaque and dental calculus in animals. Additionally, a need exists for food products, compositions, solutions or methods that are useful to effectively control, reduce, or inhibit formation of both dental plaque and calculus in animals.

### SUMMARY OF THE INVENTION

The present invention provides a method of inhibiting dental plaque and dental calculus on the teeth of a dental plaque- and calculus-forming animal, comprising contacting said teeth with a food product comprising an amount of an antimicrobial agent effective to inhibit dental plaque and an amount of a polycarboxylic acid sequestering agent effective to inhibit dental calculus. In additional embodiments of the invention, the food product may also comprise an acidulent amount of phosphoric acid. Additional embodiments of the invention provide a method of inhibiting the development of gingivitis in the oral soft tissues of dental plaque- and dental calculus-forming animals comprising contacting said teeth with a food product comprising an amount of an antimicrobial agent effective to inhibit the development of gingivitis. In one embodiment of the invention, the food product is a dry food product. In another embodiment, the food product is a moist food product. In certain embodiments of the invention, the contacting comprises mastication and/or ingestion of the food product. In one embodiment of the invention, the antimicrobial agent and the polycarboxylic acid sequestering agent are applied to the surface of the food product. In an alternative embodiment, the antimicrobial agent and the polycarboxylic acid sequestering agent are intimately admixed with the food product. In certain embodiments of the invention, the polycarboxylic acid sequestering agent is malic acid, fumaric acid, citric acid, a salt thereof, or a combination thereof. For example, the concentration of malic acid, fumaric acid, citric acid, a salt thereof, or a combination thereof, can be in the range of about 0.25-5.0% by weight of the food product. In additional embodiments of the invention, the antimicrobial agent is cetyl pyridinium chloride (CPC), chlorhexidine (CHX), a salt thereof, or a combination thereof. For example, the concentration of cetyl pyridinium chloride, chlorhexidine a salt thereof, or a combination thereof can be in the range of about 0.005-5.0% by weight of the food product. In certain embodiments of the invention, the antimicrobial agent is cetyl pyridinium chloride (CPC), chlorohexidine (CHX), a salt thereof, or a combination thereof, and wherein said polycarboxylic acid sequestering agent is malic acid, fumaric acid, citric acid, a salt thereof, or a combination thereof. In embodiments of the invention, the animal is a dog or cat, and the food product is a dog ration or a cat ration, for example, a dog snack food or a cat snack food.

In addition, the present invention provides a food product which inhibits dental plaque and dental calculus on the teeth of dental plaque- and dental-calculus forming animals upon ingestion, wherein said food product comprises an amount of an antimicrobial agent effective to inhibit dental plaque and a polycarboxylic acid sequestering agent or agents in an amount effective to inhibit dental calculus. In additional embodiments, the food product further comprises an acidulent amount of phosphoric acid. In additional embodiments of the invention the food product comprises an amount of an antimicrobial agent effective inhibit the development of gingivitis in the oral soft tissues of dental plaque- and dental calculus-forming animals wherein said food product comprises an amount of an antimicrobial agent effective to inhibit gingivitis. The food product, in additional embodiments, comprises polycarboxylic acid sequestering agents such as malic acid, fumaric acid, citric acid, a salt thereof, or a combination thereof. For example, the concentration of malic acid, fumaric acid, citric acid, a salt thereof, or a combination thereof, can be in the range of about 0.25-5.0% by weight of the food product. In other embodiments, the antimicrobial agent comprises cetyl pyridinium chloride (CPC), chlorohexidine (CHX), a salt thereof, or a combination thereof. For example, the concentration of cetyl pyridinium chloride, chlorohexidine, a salt thereof, or a combination thereof can be in the range of about 0.005-5.0% by weight of the food product. In one embodiment, the antimicrobial agent is cetyl pyridinium chloride (CPC), chlorohexidine (CHX), a salt thereof, or a combination thereof, and wherein said polycarboxylic acid sequestering agent is malic acid, fumaric acid, citric acid, a salt thereof, or a combination thereof. In certain embodiments, the animal is a cat or dog, and the food product is a dog ration or a cat ration, for example, a dog snack food or a cat snack food. In embodiments of the invention, the food product can be a dry food product or a moist food product.

Further provided is a method of inhibiting dental plaque on the teeth of a dental plaque-forming animal and inhibiting dental calculus on the teeth of a dental calculus-forming animal, comprising contacting the surface of said teeth with an aqueous solution comprising an amount of a polycarboxylic acid sequestering agent effective to inhibit dental calculus and an amount of an antimicrobial agent effective to inhibit dental plaque. Additional embodiments of the invention provide a method of inhibiting the development of gingivitis in the oral soft tissues of dental plaque- and dental calculus-forming animals comprising contacting said teeth with an aqueous solution comprising an amount of an antimicrobial agent effective to inhibit the development of gingivitis. For example, the polycarboxylic acid sequestering agent can be malic acid, fumaric acid, citric acid, a salt thereof, or a combination thereof. In certain embodiments, the concentration of malic acid, fumaric acid, citric acid, a salt thereof, or a combination thereof, is in the range of about 0.25-5.0% wt-%. In other embodiments, the antimicrobial agent is cetyl pyridinium chloride (CPC), chlorohexidine (CHX), a salt thereof, or a combination thereof. For example, the concentration of cetyl pyridinium chloride, chlorohexidine a salt thereof, or a combination thereof, is in the range of about 0.005-5.0% wt-%. In embodiments of the invention, the antimicrobial agent is cetyl pyridinium chloride (CPC), chlorohexidine (CHX), a salt thereof, or a combination thereof, and the polycarboxylic acid sequestering agent is malic acid, fumaric acid, citric acid, a salt thereof, or a combination thereof. In certain embodiments of the invention, the animal is a dog or cat.

The invention also provides an aqueous solution which inhibits dental plaque and dental calculus on the teeth of dental plaque and dental calculus-forming animals upon application to said teeth, wherein said solution comprises a polycarboxylic acid sequestering agent or agents in an amount effective to inhibit dental calculus, and wherein said solution further comprises an amount of an antimicrobial agent or agents effective to inhibit dental plaque, and to inhibit the development of gingivitis in the oral soft tissue of said animal. In additional embodiments of the invention the aqueous solution comprises an amount of an antimicrobial agent effective to inhibit the development of gingivitis in the oral soft tissues of dental plaque- and dental calculus-forming animals wherein said food product comprises an amount of an antimicrobial agent effective to inhibit gingivitis. For example, the polycarboxylic acid sequestering agent is malic acid, fumaric acid, citric acid, a salt thereof, or a combination thereof. In some embodiments, the concentration of malic acid, fumaric acid, citric acid, a salt thereof, or a combination thereof, is in the range of about 0.25-5.0% wt-%. In embodiments of the invention, the antimicrobial agent is cetyl pyridinium chloride (CPC), chlorohexidine (CHX), a salt thereof, or a combination thereof. For example, the concentration of cetyl pyridinium chloride, chlorohexidine a salt thereof, or a combination thereof, is in the range of about 0.005-5.0% wt-%. In embodiments of the invention, the polycarboxylic acid sequestering agent is malic acid, fumaric acid, citric acid, a salt thereof, or a combination thereof, and wherein said antimicrobial agent is cetyl pyridinium chloride (CPC), chlorohexidine (CHX), a salt thereof, or a combination thereof. In an embodiment of the invention, the animal is a cat or dog.

### DETAILED DESCRIPTION OF THE INVENTION

The inventor has discovered that a combination of effective amounts of one or more antimicrobial agent(s) and one or more polycarboxylic acid sequestering agent(s) effectively reduce, prevent and/or inhibit the formation of both dental plaque and dental calculus in animals, and can reduce or eliminate the incidence of gingivitis. Using the compositions, solutions and methods of the invention, the formation of dental plaque and dental calculus can be significantly reduced, inhibited or prevented all together.

As used herein, with respect to calculus or plaque, the term “inhibit” includes preventing or reducing the formation of dental calculus or plaque, as well as preventing or reducing calculus deposits or plaque deposits or rate of formation or accumulation by a measurable amount.

The present invention can be utilized to treat the oral regions of a variety of animals. Animals include mammals; cats, including domesticated cats as well as exotic animals, including lions, tigers, cougars, lynx, bobcats, mountain lions; dogs, including domesticated dogs and non-domesticated dogs, coyotes, foxes and wolves; primates, including baboons, monkeys, and apes; lemurs; bears; mink; ferrets; rabbits; and rodents. The methods and products described herein can be used to treat any animal that normally accumulates dental plaque.

Antimicrobial Agents

Antimicrobial agents are compounds or chemical agents that destroy microbes, microorganisms, or bacteria, or prevent their multiplication or growth, and/or prevent their pathogenic action. Antimicrobial agents for use in this invention include any antimicrobial agents that are suitable for use and effective in the oral cavity. Examples of antimicrobial agents that may be utilized in this invention include, but are not limited to, cetyl pyridinium chloride (CPC), triclosan, chlorohexidine gluconate (CHX), chlorhexidine acetate, chlorhexidine chloride, domiphen bromide, caprylic acid, stannous fluoride, ammonium fluoride, amine fluoride, hexachlorophene, and essential oils, including mint oil. Further examples of antimicrobial agents that may be used with this invention include those agents that are effective to prevent, inhibit or destroy some of the 300-plus species of microorganisms that cause dental plaque or gingivitis, said microorganisms include Actinomyces sp., Streptococci sp., Porphyromonas sp., Prevotella sp., Fusobacterium sp., Bacteroides sp., Actinobacillus sp., Camplyobacter sp., Eubacterium sp., Eikenella sp., and Wolinella sp., and other species.

The use of antimicrobial agents in the present invention is advantageous for a number of reasons. The biological, toxicological, and pharmacological safety of these antimicrobials (except for caprylic acid and amine fluoride) is demonstrated by their approval by the U.S. Food and Drug Administration for use in over-the-counter, professional, and/or prescription oral care products. Relatively low concentrations of antimicrobial agents can be efficacious for prevention of dental plaque formation in animals. The efficacy of antimicrobial agents in the prevention of dental plaque formation is excellent when used as described on foods also having a surface coating of phosphoric acid as a palatant and/or a surface coating of polycarboxylic acids as an anti-calculus agent. Useful antimicrobial agents are those that are compatible with commercially-prepared dry cat and dog foods that contain a polycarboxylic acid sequestering agent. Antimicrobial agents can be applied to the surface of commercially-prepared dry cat and dog foods that are surface coated with a polycarboxylic acid sequestering agent, or they can be combined with the polycarboxylic acid sequestering agent and applied to the surface of commercially prepared dry cat and dog foods that are surface coated with phosphoric acid. Antimicrobial agents can readily be incorporated or mixed into commercially-prepared moist animal food products. Finally, effective amounts of most antimicrobial agents can be used as a coating for animal foods without deleteriously altering the organoleptic and palatability characteristics of the food products

Polycarboxylic Acid Sequestering Agents

Polycarboxylic acid sequestering agents, or sequestrants, are those capable of forming soluble coordination complexes with various cations such as alkaline earth cations so as to prevent their usual precipitation from aqueous solution as insoluble deposits. The polycarboxylic acid sequestering agents useful in this invention comprise about 3-12 carbon atoms and can include about 2-3 carboxylic acid (CO₂H) groups. Polycarboxylic acid sequestering agents are malic acid (HO₂CCH₂CH(OH)CO₂H), fumaric acid (HO₂CCH═CHCO₂H), and citric acid (HO₂CCH₂C(OH)(CO₂H)CH₂CO₂H). Nontoxic water soluble, palatable salts of these acids may also be used in the present invention, such as alkali metal salts.

The use of polycarboxylic acids in the present invention is advantageous for a number of reasons. The biological, toxicological, and pharmacological safety of these polycarboxylic acids upon animal ingestion is demonstrated by their acceptance as GRAS by the U.S. Food and Drug Administration. Relatively low concentrations of polycarboxylic acids can be efficacious for prevention of dental calculus formation in animals. The efficacy of polycarboxylic acids in the prevention of dental calculus formation is markedly superior as compared to prior art crystal growth inhibitors, when both are used as described herein below, on foods also having a surface coating of phosphoric acid as a palatant. Unlike hexametaphosphate, polycarboxylic acids are compatible with commercially-prepared dry foods for cats and dogs that are surface coated with phosphoric acid. Polycarboxylic acid sequestering agents can readily be incorporated or mixed into commercially-prepared moist animal food product. Additionally, the use of effective amounts of polycarboxylic acids as a coating for animal foods does not deleteriously alter the organoleptic and palatability characteristics of the food products.

Polyphosphate Sequestering Agents

Commonly-used antimicrobial agents may not be compatible with various polyphosphates that are commonly used to prevent or reduce the formation of dental calculus by sequestering calcium and other cations to form soluble complexes that are swallowed or expectorated rather than form calcium precipitates within dental plaque. The types of polyphosphates that are not compatible with antimicrobial agents such as cetyl pyridinium chloride and chlorhexidine gluconate include sodium hexametaphosphate and various soluble pyrophosphate salts such as sodium acid pyrophosphate, potassium acid pyrophosphate, tetrasodium pyrophosphate, and tetrapotassium pyrophosphate. The present inventor discovered that combinations of polyphosphates, such as sodium hexametaphosphate, and antimicrobial agents, such as cetyl pyridinium chloride or chlorhexidine gluconate, immediately form insoluble precipitates. Clinical studies using these latter combinations indicate that the combinations have no effect on the formation of dental plaque (i.e., loss of antimicrobial activity), nor any effect upon the formation of dental calculus.

Therapeutic Compositions for Oral Application

In one embodiment, the composition of the invention is an aqueous solution containing an effective amount of at least one antimicrobial agent and an effective amount of at least one polycarboxylic acid sequestering agent. The oral compositions of the present invention include but are not limited to solutions in the form of rinses, suspensions, emulsions, drinks, drink additives, mouthwashes, gels, powders, creams, or lotions. In an embodiment, the oral composition is an aqueous solution which is topically administered, usually in the form of a solution for oral use to prevent, reduce, or inhibit the formation of dental plaque and dental calculus, as well as gingivitis. In one embodiment, the solution contains about 0.25-10% wt/vol polycarboxylic acid sequestering agent, for example, 0.025-5.0% wt/vol, and about 0.005-5.0% wt/vol antimicrobial agent, for example, 0.005-1.5% wt/vol antimicrobial agent. Forms of the solution for use in the present invention include about 0.5-3.0% wt/vol polycarboxylic acid sequestering agent and about 0.025-1.5% wt/vol antimicrobial agent. The composition of the present invention may be formulated with one or more pharmaceutically acceptable carriers therefore and, optionally, other therapeutic and/or prophylactic ingredients. The cations and carrier(s) must be “acceptable” in the sense of being compatible with the other ingredients of the formulation and not deleterious to the recipient thereof, i.e., they do not produce an adverse, allergic, or other untoward reaction when administered to an animal at appropriate levels.

The oral composition can be given as a water-based formulation, a solution, a suspension or an emulsion. Application of the composition can include, but is not limited to, topical administration by swabbing directly the teeth. The oral composition can also be in the gel form, and be directly applied to the teeth by swabbing directly on the teeth or by placing the gel in the dental tray which is inserted into the mouth of the animal for an appropriate amount of time to allow the active ingredients to act upon the tooth surface and surrounding soft tissue.

The composition can be applied to the teeth or oral cavity of an animal via oral rinse or mouthwash, or an ingestible drink. For either oral rinse or ingestible drink, a preferable adjuvant can be chosen from among a number of non-cariogenic carbohydrates, such as xylose or sorbitol. A dry mixture of carbohydrate carrier and an effective amount of antimicrobial agent and polycarboxylic acid sequestering agent can be provided, for reconstitution with water, juice, or other liquid to form a suspension or a drink.

It will be appreciated that the amount of the antimicrobial agent and polycarboxylic acid sequestering agent required for use in treatment will vary with the route of administration, the severity of the condition being treated and the age and condition of the animal.

Food Product Composition

Exemplary food products for use in this invention include rations and snack foods for dogs and cats. Said food products contain an amount of polycarboxylic acid and an amount of an antimicrobial agent either applied onto the surface of, or mixed into, the food products.

In one embodiment of the present invention, the polycarboxylic acid sequestering agent is about 0.05-10% by dry weight of the food product, and the antimicrobial agent is about 0.001-5.0% by dry weight of the food product. In another embodiment of the present invention, the polycarboxylic acid sequestering agent is about 0.25-5.0% by dry weight of the food product and the antimicrobial agent is about 0.005-5.0% by dry weight of the food product. In another embodiment of the present invention, the polycarboxylic acid sequestering agent is about 0.25-3.0% by dry weight of the food product and the antimicrobial agent is about 0.005-2.0% by dry weight of the food product. In another embodiment of the present invention, the polycarboxylic acid sequestering agent is about 0.25-3.0% by dry weight of the food product and the antimicrobial agent is about 0.005-1.5% by dry weight of the food product. The polycarboxylic acid sequestering agent is combined with the antimicrobial agent in a single aqueous solution, applied to the dry food product as a surface coating, and dried. The present invention provides food products referred to herein as rations and snack foods. As used herein, the term “dog ration” or “cat ration” for purposes of this invention denotes an allowance or measure of food to sustain the daily dietary and nutritional needs of an average domesticated dog or cat, respectively. Dog or cat rations may be formulated so as to be dry or moist. Dry rations typically contain a minimum of about 5-50% crude protein, a minimum of about 0.5-25% crude fat, a maximum of about 1-10% crude fiber, and a maximum of about 1-30% moisture. Moist rations typically contain a minimum of about 0.5-40% crude protein, a minimum of about 0.5-25% crude fat, a minimum of about 0.5-15% crude fiber, a maximum moisture content of about 60-95%, a maximum of about 0.1-20% ash, a minimum of about 0.001-5.0% taurine, and water in an amount sufficient for processing.

As used herein, the terms “dog snack food” and “cat snack food” are used to refer to food items which are intended to augment or supplement a ration, to remove dental plaque through the normal mastication process, or simply entertain or amuse a dog or cat, respectively. Typical snack foods contain a minimum of about 5-45% crude protein, a minimum of about 0.5-25% crude fat, a maximum of about 0.25-20% crude fiber, and a maximum of about 0.5-50% moisture. The forms of snack foods vary greatly, and may encompass shapes such as biscuits, crackers, rings, kernels, wafers, cylindrical sticks or bones.

The above-described food products contain components of starch or carbohydrates, animal or fish proteins, animal or fish by-products, coloring agents, flavoring agents and palatants, sources of fat, stabilizers, emulsifiers, texturizers, thickening and binding agents, leavening agents, preservatives, nutritional factors, dietary components, and vitamin supplements.

The present rations and snack foods can contain an amount of carbohydrate or starch. Useful carbohydrate or starch components include cereals and grains, including ground yellow corn, ground white corn, puffed corn, modified corn starch, corn gluten meal, rice, rice flour, crisp rice, brewers rice, rice flour, barley, ground barley, soybean, soybean meal, soy flour, oats, oat gluten, oat flour, oatmeal, tapioca, tapioca starch, wheat, wheat bran, wheat flour, bleached wheat flour, unbleached wheat flour, wheat gluten, defatted wheat germ, toasted whole wheat, malt, and whole wheat flour.

Forms of animal or fish protein may be present in the present rations and snack foods. For dogs and cats, such proteins include by-products and by-product meals, digests and bone meal, from beef, chicken, pork, veal, turkey, poultry, lamb, or fish. Protein forms for use in human snack foods generally comprise animal proteins, including beef, chicken, pork, veal, turkey, poultry or lamb protein, as well as milk solids, such as sodium caseinate and whey.

Leavening agents can be present in rations and snack foods. Leavening agents include sodium bicarbonate and monocalcium phosphate.

Coloring agents can also be included in the present rations and snack foods. Useful coloring agents include caramel coloring, yellow dyes #5 & #6, titanium dioxide, and iron oxide.

Fat is included in the present rations and snack foods. Sources of fat in the present rations and snack foods include vegetable oil, canola oil, safflower oil, sunflower oil, cottonseed oil, soybean oil, peanut oil, olive oil, and butter. These oils may be present in human snack foods in the hydrogenated or partially hydrogenated form.

The present rations and snack foods can incorporate palatants and flavoring agents, to enhance the taste and flavor of the products. Palatants and flavoring agents include salt, natural flavors, artificial flavors, spices, yeast, brewers dried yeast, yeast extract, fermented soy, sugars, corn sweeteners, nuts, sesame seeds and phosphoric acid.

Stabilizers, protective colloids, emulsifiers, texturizers, thickening and binding agents can also be included in the present snack foods and rations, including guar gum, locust bean gum, carrageenan, iron oxide, powdered cellulose, modified cellulose, sodium alginate, xanthan gum, and propylene glycol.

The present rations and snack foods can contain preservatives and antioxidants which inhibit biodegradation, fermentation, mold growth and infestation by other organisms, such as rodents or insects. Useful preservatives and antioxidants include ethoxyquin, propylene glycol, parabens and potassium sorbate.

Nutritional factors, dietary supplements, and vitamin supplements can also be contained in the present rations and snack foods. Nutritional factors and dietary supplements include zinc sulfate, ferrous sulfate, potassium chloride, calcium carbonate, choline chloride, niacin, copper sulfate, manganese sulfate, calcium pantothenate, folic acid, potassium iodide, biotin, sodium selenite, DL methionine, ferrous sulfate, dicalcium phosphate, niacin, and copper sulfate. Vitamin supplements include vitamins A, C, E, B12, D3, thiamine mononitrate and thiamine hydrochloride (vitamin B1), riboflavin supplement (Vitamin B2), pyridoxine hydrochloride (Vitamin B6), and menadione sodium bisulfite complex (source of vitamin K activity).

For dogs and cats, the present food products, rations and snack foods may also contain compounds as palatants, such as phosphoric acid.

### EXAMPLE 1

### Analysis of Antimicrobial Activity of Selected Mixtures of Anti-Calculus and Antimicrobial Compounds

This study was conducted to determine whether anti-calculus agents inhibit the antimicrobial activity of cetyl pyridium chloride (CTC) and chlorhexidine (CHX).

Experimental Design: TSA agar plates were individually swabbed with fresh 24 h broth cultures of Streptococcus mutans (TH16), Streptococcus parasanguis (FW213) and Actinomyces viscosus (AV) to provide a confluent lawn of bacterial growth. Five minutes after swabbing the plates (to allow the inoculum to absorb into the agar), 20 ul of each test specimen (see below) was placed onto designated places on the plates and allowed to absorb into the agar. The plates were incubated agar-side up at 37° C. in 5% CO₂ for 48 h. Plates were examined at 24 and 48 h for zones of inhibition of growth for each bacterial species. The diameter of each zone was measured in mm. The zones of inhibition of each mixture (anti-calculus and antimicrobial agents) were compared to the zones of each agent separately. If the zones of the mixtures were the same size (or larger) than the inhibition zones from the antimicrobial agent alone then there was no inhibition of antimicrobial activity by the anti-calculus agent.

Test Specimens: There were 12 different samples/test specimens used in this experiment. Test groups included (concentrations are wt. %): cetyl pyridium chloride (CPS; commercially available from ICN Biomedicals, Inc., Aurora, Ohio) at a concentration of 0.05%; CPC at 0.05%+1.0% malic acid; CPC at 0.05%+1% fumaric acid; CPC at 0.05%+1% citric acid; chlorhexidine (CHX; commercially available ICN Biomedicals, Inc., Aurora, Ohio) at 0.1% alone; CHX at 0.1%+1% malic acid; CHX at 0.1%+1% fumaric acid; CHX at 0.1%+1% citric acid; 1% malic acid; 1% fumaric acid; 1% citric acid.

Results: See Table 1 (below). This study was designed to determine whether or not the polycarboxylic acids (anti-calculus agents) altered the activity of the anti-microbial agents. Decreases in the width of the zone of inhibition when the polycarboxylic acid is combined with the antimicrobial agent, as compared to the width of the zone of inhibition for the antimicrobial agent alone, is an indication that the polycarboxylic acid reduced the activity of the anti-microbial agent. Differences in the width of the zone of inhibition of ±3 mm are not considered to be significant. As may be seen from the Table, the activity of cetyl pyridinium chloride was not altered by combining it with any of the polycarboxylic acids tested. Similarly, the activity of chlorhexidine gluconate against two of the microorganisms (S. mutans and S. parasanguis) was not altered by combining it with the polycarboxylic acids. However, the activity of chlorhexidine against A. viscosus was decreased by the presence of fumaric acid. Collectively these data indicate the compatibility of these two antimicrobial agents with these polycarboxylic acids.

TABLE 1
Zones of Inhibition of Growth in mm

| Treatment | TH16 | FW213 | AV |
|---|---|---|---|
| dH₂O | 0 | 0 | 0 |
| CPC alone | 7 | 9 | 14 |
| CHX alone | 6 | 13 | 20 |
| M (malic acid) | Weak* | 0 | 0 |
| M + CPC | 8 | 8 | 15 |
| M + CHX | 6 | 12 | 17 |
| C (citric acid) | Weak* | 0 | 0 |
| C + CPC | 6 | 9 | 12 |
| C + CHX | 6 | 12 | 17 |
| F (fumaric acid) | Weak* | 0 | 0 |
| F + CPC | 6 | 9 | 13 |
| F + CHX | 7 | 12 | 15 |

Note:
CPC is cetyl pyridinium chloride
Note:
CHX is chlorhexidine gluconate
*Weak = very slight activity; <1 mm

### Animal Studies

Description of Animal Studies

The invention will be further described by reference to the following detailed examples that were designed to evaluate measures and agents for the prevention of dental plaque and dental calculus formation.

A group of 31 or 35 colony-bred adult female cats and 24 beagle dogs was used. The animals were continuously housed in stainless steel cages in an AAALAC-accredited facility in the Indiana University School of Dentistry. To establish the normal rate of calculus formation for each animal, a thorough dental prophylaxis (cleaning) was performed on each animal to remove all existing calculus and other exogenous deposits (plaque, pellicle, debris) from all of their teeth. The cats were then provided with a conventional dry chow diet, Purina Cat Chow, which is a commercially-available, nutritionally balanced cat ration. Kal Kan Whiskas® dry diet contains 0.5 weight-% phosphoric acid. The cats were fed an amount of diet on the basis of body weight (about 15.5 g/kg body weight). This feeding was provided once daily throughout a 28-day test period.

The dogs were provided with a conventional dry diet, Purina Dog Chow, which is a nutritionally balanced, commercially-available dog ration. Waltham Dry Adult Conditioning Formula® brand diet contains 0.25 weight-% phosphoric acid. The dogs were fed an amount of diet on the basis of body weight (about 23 g/kg body weight). This feeding was similarly provided once daily throughout a 28-day test period. Fresh tap water was freely available to all animals throughout the studies.

At the conclusion of the test period, the cats were anesthetized and buccal (cheek) surfaces of their posterior teeth were clinically examined for the presence of calculus, plaque and gingivitis in the reverse order. For these examinations the tooth surface is visually divided into three segments: mesial one-third, middle third, and distal one-third. The presence of gingivitis was always graded first and was assessed clinically adjacent to the three segments of the buccal surface of each tooth using a conventional periodontal probe and the following numerical scale: 0=no gingivitis; 1=incipient or very mild gingivitis, i.e. red and/or swollen gingival but no bleeding on probing; 2=mild gingivitis observed as red and swollen gingival with delayed bleeding on gentle probing; 3=moderate gingivitis observed as red and swollen gingival with immediate bleeding upon gentle probing; and 4=severe gingivitis observed as ulceration and/or spontaneous hemorrhaging and profuse bleeding upon gentle probing. The results were summarized for each animal by summing the three scores of each tooth segment to obtain the individual tooth score, summing the tooth scores and dividing by the number of teeth graded. The presence of dental plaque was assessed clinically by disclosing the plaque with an aqueous solution containing 1.5% D&C Red #28, removing the excess disclosant by spraying with tap water, and then determining the amount of plaque present on the occlusal and gingival halves of the buccal surface of each tooth using the following numerical scale: 0=no observable plaque; 1=scattered plaque covering <24% of the tooth surface; 2=plaque covering between 25 and 49% of the tooth surface; 3=plaque covering between 50 and 74% of the tooth surface; and 4=plaque covering 75% or more of the tooth surface. The plaque intensity or thickness was assessed for each area using the following subjective numerical scale: 1=light or thin layer of plaque; 2=moderate thickness of about 1 mm; and 3=heavy or thick layer of plaque in excess of 1 mm in thickness. The results of the assessments were summarized for each animal by multiplying the plaque coverage score by the intensity factor for each occlusal and gingival segment of each tooth, summing the scores for the occlusal and gingival segments to obtain an individual tooth score, and summing the individual tooth scores and dividing by the number of teeth graded. Following the dental plaque examinations the disclosed plaque was removed by brushing the tooth surfaces with a toothbrush and tap water leaving the underlying calculus. Calculus was assessed by the Warrick-Gorrel method. For this examination, the buccal surface of each tooth is divided vertically into thirds and each third is assigned a numerical score of 0 to 4 based on calculus, plaque, and gingivitis coverage as follows: 0=none; 1=less than 24% coverage; 2=25 to 49% coverage; 3=50 to 74% coverage; 4=greater than 75% coverage. The density or thickness of the calculus deposit on each tooth segment was graded using a periodontal probe calibrated in 1 mm increments and the following numerical scale: 1=light deposit, thin and smooth, and <0.5 mm in thickness; 2=moderate thickness with porosity, frequent ledging and a thickness greater than 0.5 mm but less than 1.0 mm; 3=heavy deposit with extensive porosity, bumpy texture and a thickness greater than 1.0 mm. The individual tooth surface score is obtained by multiplying the coverage and density scores for each of the three tooth segments and summing the scores of the 3 segments of the tooth surface. A mean tooth surface score for each cat was obtained by summating the individual tooth surface scores and dividing by the number of teeth graded

The method used to determine the presence of calculus, plaque, and gingivitis formation in the dogs differed from that used in cats only in the number of teeth examined. At the conclusion of the test period, the dogs were anesthetized and buccal (cheek) surfaces of their posterior teeth were clinically examined for the presence of calculus, plaque, and gingivitis. The dogs were first examined for the presence of gingivitis using the procedures described previously for cats. The presence of disclosed dental plaque then was assessed by using essentially the same procedure as described previously for cats. Upon completion of the plaque assessments the disclosed plaque was removed by brushing the teeth with a toothbrush and tap water leaving the underlying calculus. The presence of dental calculus was assessed using a grading system similar to the Volpe-Manhold index used in human clinical trials of anti-calculus agents and modified by Warrick and Gorrel to increase sensitivity. For this examination, each tooth surface was visually divided into three tooth segments (mesial, middle, distal) as described previously, and each segment was graded for the presence and the density (thickness) of the deposit as described previously. The individual tooth surface calculus scores were summated and then divided by the number of tooth surfaces graded to obtain a mean tooth surface score for each dog in the same manner as described above for cats.

The calculus, plaque, and gingivitis scores obtained during the baseline/pre-test period were considered to reflect the normal rate of calculus, plaque, and gingivitis formation for each animal and were used to stratify the animals into groups for all subsequent tests. For example, for a planned 4-group test, the individual animal scores were ranked from the highest to lowest, stratified into blocks of 4 (i.e., the 4 highest scores comprised the first block, the next 4 highest scores comprised the second block, etc.), and then randomly assigned within blocks to each of the 4 groups (so-called randomized block procedure). Thus, each group was comprised of randomly-selected representative animals from each block.

In all experimental studies, the clinical examinations were performed without knowledge of treatment groups by an examiner not otherwise involved in group assignment or daily feeding regimens. Further, the sequence of animals subjected to these examinations was randomized to minimize systematic bias due to examiner fatigue, etc.

For each experiment, the animals were given a thorough dental prophylaxis immediately prior to initiation of the study. The animals were divided into the desired number of groups using the previously-described randomized block assignment procedure. The designated feeding regimens were provided beginning with the evening feeding following the prophylaxis, for 4 consecutive weeks. Food consumption was monitored for each animal at each feeding to assure that experimental differences were not confounded by differences in food consumption.

At the end of the 28-day test period, the animals were anesthetized and examined for dental calculus, dental plaque, and gingivitis formation as previously described. The data were subsequently analyzed using conventional biostatistical procedures, which routinely involved an analysis of variance (ANOVA) with the Newman-Keuls test procedure used to identify statistically significant (p is less that 0.05) intergroup differences. Between experiments the cats were maintained on the commercially-available Purina Cat Chow for a one-week (minimum) washout period to eliminate any possible carryover effects from the previous test and the dogs were similarly maintained on a commercially-available Purina Dog Chow for the same amount of time, for the same purpose.

### EXAMPLE 2

A study was conducted to determine the effect of cetyl pyridinium chloride and chlorhexidine gluconate in combination with malic acid upon the formation of dental plaque, calculus and gingivitis in cats. The cats were maintained on a conventional dry diet (Purina Cat Chow) as noted earlier and stratified into 3 groups. The active agents were applied to the teeth as aqueous solutions (0.5 ml per quadrant) 2-3 hours following the feeding of the basal diet each morning. The treatment regimens were: (a) deionized water control; (b) 3.0% malic acid+0.2% chlorhexidine gluconate; and (c) 3.0% malic acid+0.075% cetyl pyridinium chloride. The results of the study are summarized in the following table.

TABLE 2
Effect of Two Different Anti-calculus and Antimicrobial Systems
Upon Plaque, Calculus and Gingivitis Formation in Cats

| Agent | Number | Calculus | Plaque | Gingivitis |
|---|---|---|---|---|
| Tested | of Cats | Score | Score | Score |
| None | 20 | 0.81 ± 0.12 | 6.04 ± 0.23 | 0.72 ± 0.04 |
| (Control) | | | | |
| Malic + | 21 | 0.41 ± 0.09* | 4.84 ± 0.28 | 0.59 ± 0.03 |
| CPC | | (49.4%) | (19.9%) | (18.1%) |
| Malic + | 21 | 0.35 ± 0.07 | 4.66 ± 0.30 | 0.56 ± 0.04 |
| CHX | | (56.9%) | (22.8%) | (22.2%) |

*Mean ± Standard error of the mean

The results of this study indicate that both of these combinations of anti-microbial and anti-calculus agents exerted a significant protective effect on all three conditions of interest, i.e., dental plaque, calculus and gingivitis.

### EXAMPLE 3

A series of studies was conducted in cats to determine the most effective antimicrobial agents for reducing the formation of dental plaque. Because of the limited number of available animals (31 cats) and the need for group sizes of at least 10 to account for biological variation, each study compared an individual agent to cetyl pyridinium chloride. The treatments were applied as rinses with 4-week test periods using the same procedures as described for Example 2. The amount of plaque present at the conclusion of the test period was graded clinically using the numerical scale described previously. The results of these studies indicated that only two of the antimicrobial agents tested consistently resulted in reductions of dental plaque of 25-40%; these agents were cetyl pyridinium chloride and chlorhexidine gluconate. Triclosan and caprylic acid resulted in lesser reductions in the formation of dental plaque of 14.8 and 10.2%, respectively, while no reduction was observed with rinses containing stannous fluoride. Based upon the results of these studies cetly pryidinium chloride and chlorhexidine gluconate were identified as antimicrobial agents useful in embodiments of the invention.

### EXAMPLE 4

The compatibility of three antimicrobial agents (tricolsan, chlorohexidine gluconate and cetyl pyridinium chloride) with three anti-calculus, polycarboxylic acid sequestering agents (malic acid, citric acid, and fumaric acid) was evaluated.

Each of the antimicrobial agents was separately mixed with each of the anti-calculus sequestering agents. This procedure involved preparing fresh solutions of the various agents at concentrations within the range of possible use; these concentrations were: 0.1% triclosan; 0.12% chlorhexidine gluconate; 0.05% cetyl pyridinium chloride; 3% malic acid; 3% citric acid; and 3% fumaric acid. One antimicrobial agent, triclosan, immediately formed a precipitate with malic acid. The other combinations remained as clear solutions. Thus, only the combination of malic acid and triclosan was considered to lack compatibility for further use.

### EXAMPLE 5

A study was conducted to compare the effectiveness of the combination of an antimicrobial agent and an anticalculus agent on the teeth of cats when the agents were applied as a coating to a conventional commercially available cat food (Meow Mix) and tested as a cat treat product. To prepare the experimental test product 0.10 ml of an aqueous solution containing 3.0% (wt-vol %) malic acid and 0.125% (wt-vol %) chlorhexidine gluconate was applied to each kiblet and the kiblets dried. The 35 cats in the colony were stratified as described earlier into 3 groups and provided the following experimental snack regimens: Group A, test kiblets; Group B, placebo kiblets (treated with 0.1 ml of deionized water and dried); and Group C, no treat. Using the previously described procedures, the teeth of the cats were cleaned with a dental prophylaxis at the initiation of the study and the animals were provided tap water and a basal diet (15.5 g/kg body weight less the amount of food provided as a treat in groups A and B). Three hours later the cats in Groups A and B were provided 3.0 g (12-13 kiblets) of their designated test product as a treat. This daily regimen was maintained for 4 weeks at which time the cats were anesthetized and examined for calculus and plaque.

TABLE 3
Effect of Treat Product Coated with Malic Acid and
the Antimicrobial Agent Chlorhexidine Solutions on
Calculus, Plaque, and Gingivitis Formation in Cats

| | Number | | | |
|---|---|---|---|---|
| Treatment | of Cats | Calculus | Plaque | Gingivitis |
| No Treat | 12 | 0.71 | 6.40 | 0.60 |
| Placebo Treat | 12 | 0.71 | 5.84 | 0.55 |
| | | (0%)* | (9%)* | (8%)* |
| Malic Acid + | 11 | 0.57 | 5.79 | 0.62 |
| Chlorhexidine | | (20%)* | (10%)* | (0%)* |

*Percent difference or percent reduction from the control treatment

These data indicate that the combination of malic acid and an antimicrobial agent (chlorhexidine gluconate) added as a coating to a commercially available cat food resulted in reductions in the formation of dental calculus and dental plaque as compared to either the placebo product or the control (no treat) regimen. Although no benefit on gingivitis was observed in this instance, previous studies in our facility have indicated that longer test periods are often necessary to observed the maximum impact upon gingivitis (Kantmann et al., Conference Proceedings, Annual Veterinary Dental Forum, pp 373-376, 2001).

All publications, patents (including U.S. Pat. No. 6,080,419) and patent documents are incorporated by reference herein, as though individually incorporated by reference. The invention has been described with reference to various specific and preferred embodiments and techniques. However, it should be understood that these specific and preferred embodiments and techniques are non-limiting, and that many variations and modifications may be made while remaining within the spirit and scope of the invention.

What is claimed is:

1. A method of inhibiting dental plaque and dental calculus on the teeth of a dental plaque- and calculus-forming animal, comprising contacting said teeth with a food product comprising:
(a) an amount of an antimicrobial agent effective to inhibit dental plaque; and
(b) an amount of a polycarboxylic acid sequestering agent effective to inhibit dental calculus.

2. The method of claim 1 wherein said food product is a dry food product.

3. The method of claim 1 wherein said food product is a moist food product.

4. The method of claim 2 wherein said contacting is by ingestion of the food product.

5. The method of claim 3 wherein said contacting is by ingestion of the food product.

6. The method of claim 1 wherein the antimicrobial agent and the polycarboxylic acid sequestering agent are applied to the surface of the food product.

7. The method of claim 1 wherein the antimicrobial agent and the polycarboxylic acid sequestering agent are intimately admixed with the food product.

8. The method of claim 1 wherein said polycarboxylic acid sequestering agent is malic acid, fumaric acid, citric acid, a salt thereof, or a combination thereof.

9. The method of claim 8 wherein malic acid, fumaric acid, citric acid, a salt thereof, or a combination thereof, is present at concentration of about 0.25-5.0% by weight of the food product.

10. The method of claim 1 wherein said antimicrobial agent is cetyl pyridinium chloride (CPC), chlorhexidine (CHX), a salt thereof, or a combination thereof.

11. The method of claim 10, wherein the cetyl pyridinium chloride, chlorhexidine, a salt thereof, or a combination thereof is present at concentration of about 0.005-5.0% by weight of the food product.

12. The method of claim 1 wherein said antimicrobial agent is cetyl pyridinium chloride (CPC), chlorohexidine (CHX), a salt thereof, or a combination thereof, and wherein said polycarboxylic acid sequestering agent is malic acid, fumaric acid, citric acid, a salt thereof, or a combination thereof.

13. The method of claim 1 wherein said animal is a dog or cat.

14. The method of claim 1 wherein said food product is a dog ration or a cat ration.

15. The method of claim 1 wherein said food product is a dog snack food or a cat snack food.

16. A food product which inhibits dental plaque and dental calculus on the teeth of dental plaque- and dental calculus-forming animals upon ingestion, wherein said food product comprises:
(a) an amount of an antimicrobial agent effective to inhibit dental plaque; and
(b) a polycarboxylic acid sequestering agent or agents in an amount effective to inhibit dental calculus.

17. The food product of claim 16 wherein said polycarboxylic acid sequestering agent is malic acid, fumaric acid, citric acid, a salt thereof, or a combination thereof.

18. The food product of claim 17 wherein the malic acid, fumaric acid, citric acid, a salt thereof, or a combination thereof, is present at a concentration of about 0.25-5.0% by weight of the food product.

19. The food product of claim 18, wherein said antimicrobial agent is cetyl pyridinium chloride (CPC), chlorohexidine (CHX), a salt thereof, or a combination thereof.

20. The food product of claim 19, wherein the cetyl pyridinium chloride, chlorohexidine, a salt thereof, or a combination thereof is present at a concentration of about 0.005-2.0% by weight of the food product.

21. The food product of claim 16 wherein said antimicrobial agent is cetyl pyridinium chloride (CPC), chlorohexidine (CHX), a salt thereof, or a combination thereof, and wherein said polycarboxylic acid sequestering agent is malic acid, fumaric acid, citric acid, a salt thereof, or a combination thereof.

22. The food product of claim 16 wherein said animal is a cat or dog.

23. The food product of claim 16 which is a dog ration or a cat ration.

24. The food product of claim 16 which is a dog snack food or a cat snack food.

25. The food product of claim 16 which is a dry food product.

26. The food product of claim 16 which is a moist food product.

27. A method of inhibiting dental plaque and dental calculus on the teeth of a dental plaque- and dental calculus-forming animal, comprising contacting the surface of said teeth with an aqueous solution comprising:
(a) an amount of a polycarboxylic acid sequestering agent effective to inhibit dental calculus; and
(b) an amount of an antimicrobial agent effective to inhibit dental plaque.

28. The method of claim 27 wherein said polycarboxylic acid sequestering agent is malic acid, fumaric acid, citric acid, a salt thereof, or a combination thereof.

29. The method of claim 28 wherein the concentration of malic acid, fumaric acid, citric acid, a salt thereof, or a combination thereof, is about 0.25-5.0% wt-%.

30. The method of claim 27 wherein said antimicrobial agent is cetyl pyridinium chloride (CPC), chlorohexidine (CHX), a salt thereof, or a combination thereof.

31. The method of claim 30, wherein the cetyl pyridinium chloride, chlorohexidine, a salt thereof, or a combination thereof, is present at a concentration of about 0.005-2.0% wt-%.

32. The method of claim 27 wherein said antimicrobial agent is cetyl pyridinium chloride (CPC), chlorohexidine (CHX), a salt thereof, or a combination thereof, and wherein said polycarboxylic acid sequestering agent is malic acid, fumaric acid, citric acid, a salt thereof, or a combination thereof.

33. The method of claim 27 wherein said animal is a dog or cat.

34. An aqueous solution which inhibits dental plaque and dental calculus on the teeth of dental plaque- and dental calculus-forming animals upon application to said teeth, wherein said solution comprises:
(a) a polycarboxylic acid sequestering agent or agents in an amount effective to inhibit dental calculus; and
(b) wherein said solution further comprises an amount of an antimicrobial agent effective to inhibit dental plaque.

35. The solution of claim 34 wherein said polycarboxylic acid sequestering agent is malic acid, fumaric acid, citric acid, a salt thereof, or a combination thereof.

36. The solution of claim 35 wherein the malic acid, fumaric acid, citric acid, a salt thereof, or a combination thereof, is present at a concentration of about 0.25-5.0% wt-%.

37. The solution of claim 34, wherein said antimicrobial agent is cetyl pyridinium chloride (CPC), chlorohexidine (CHX), a salt thereof, or a combination thereof.

38. The solution of claim 34 wherein said polycarboxylic acid sequestering agent is malic acid, fumaric acid, citric acid, a salt thereof, or a combination thereof, and wherein said antimicrobial agent is cetyl pyridinium chloride (CPC), chlorohexidine (CHX), a salt thereof, or a combination thereof.

39. The solution of claim 34 wherein said animal is a cat or dog.
